(19) United States
(12) Patent Application Publication
(10) Pub. No.: US 2008/0242954 A1
(43) Pub. Date: Oct. 2, 2008

(54) IMAGE PRESENTATION APPARATUS, IMAGE PRESENTATION METHOD AND PROGRAM

(75) Inventors: Masayuki NAYA, Kanagawa (JP); Shuji ONO, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/056,794

(22) Filed: Mar. 27, 2008

(30) Foreign Application Priority Data
Mar. 30, 2007 (JP) 2007-094279

(51) Int. Cl. A61B 5/00

(52) U.S. Cl. 600/300

(57) ABSTRACT

An image presentation apparatus is provided that includes a display section that sequentially displays a plurality of images, a biosignal acquisition section that acquires a signal value of a biosignal indicating a biological response of an observer watching the image displayed on the display section, and a display controlling section that controls a timing at which an image subsequent to the image being displayed on the display section is displayed on the display section based on the signal value acquired by the biosignal acquisition section.

### CROSS REFERENCE TO RELATED APPLICATION

The present application claims priority from a Japanese Patent Application No. 2007-094279 filed on Mar. 30, 2007, the contents of which are incorporated herein by reference.

### BACKGROUND

1. Technical Field

The present invention relates to an image presentation apparatus, an image presentation method, and a program and, more particularly, the present invention relates to an image presentation apparatus, an image presentation method, and a program for presenting an image to an observer observing the image to control a mental state of the observer.

2. Related Art

In the present invention, control of a mental state means overcoming of weaknesses, relaxation, enhancement of concentration, stimulation of the brain, and the like. Conventional technology for realizing such control of a mental state has been proposed. Specifically, an anxiety treatment apparatus provided with a relaxation apparatus that promotes generation of alpha waves to relax a patient and a virtual reality apparatus that presents the patient with images or sounds of a target fear has been proposed (see, e.g., Japanese Patent Application Publication No. hei 11-155955). The anxiety treatment apparatus described in Patent Document 1 treats the anxiety of the patient by operating the relaxation apparatus and the virtual reality apparatus together with each other.

In the anxiety treatment apparatus described in Japanese Patent Application Publication No. hei 11-155955, however, in a case where the fear of the patient becomes too great or the anxiety of the patient becomes too much to bear, an operator can stop the images or sounds of the target fear generated by the virtual reality apparatus. Subsequently, the operator can operate the relaxation apparatus to induce generation of alpha waves included in the brain waves of the patient. Accordingly, there is a case where presentation of the images and sounds appropriate for treating the anxiety of the patient cannot automatically correspond to the psychological condition and anxiety of the patient.

### SUMMARY

Therefore, it is an object of an aspect of the present invention to provide an image presentation apparatus, an image presentation method and a program that are capable of overcoming the above drawbacks accompanying the related art. The above and other objects can be achieved by combinations described in the independent claims. The dependent claims define further advantageous and exemplary combinations of the present invention.

According to a first aspect related to the innovations herein, one exemplary apparatus may include an image presentation apparatus. The image presentation apparatus includes a display section that sequentially displays a plurality of images, a biosignal acquisition section that acquires a signal value of a biosignal indicating a biological response of an observer watching the image displayed on the display section, and a display controlling section that controls a timing at which an image subsequent to the image being displayed on the display section is displayed on the display section based on the signal value acquired by the biosignal acquisition section.

Furthermore, the image presentation apparatus may include a comparison value storage section that stores a predetermined comparison value of a biosignal and a biosignal value comparison section that compares the signal value acquired by the biosignal acquisition section to the comparison value. In the image presentation apparatus having such a configuration, the display controlling section may control the timing at which the subsequent image is displayed on the display section based on a comparison result by the biosignal value comparison section.

Furthermore, in the image presentation apparatus having the configuration described above, the display controlling section may display the subsequent image on the display section in a case where the signal value acquired by the biosignal acquisition section reaches the comparison value.

In the image presentation apparatus having the configuration described above, the comparison value storage section may store a first comparison value of a biosignal and a second comparison value of a biosignal, which is greater than the first comparison value, and the display controlling section may control the timing at which the subsequent image is displayed on the display section to keep the signal value acquired by the biosignal acquisition section between the first comparison value and the second comparison value.

In the image presentation apparatus having the configuration described above, the display controlling section may display the subsequent image on the display section in a case where the signal value acquired by the biosignal acquisition section is outside of a range between the first comparison value and the second comparison value. Furthermore, the display controlling section may display the subsequent image on the display section in a case where the signal value acquired by the biosignal acquisition section is outside of the range between the first comparison value and the second comparison value for a predetermined length of time.

In the image presentation apparatus having the configuration described above, the comparison value storage section may store a comparison value for every period of time that passes since the image begins to be displayed on the display section. In such a case, the biosignal value comparison section may compare the signal value acquired by the biosignal acquisition section to the comparison value corresponding to the amount of time that has passed since the image began to be displayed on the display section.

In the image presentation apparatus having the configuration described above, the display controlling section may have an interval period, which is a period occurring after display of the image is finished and before the subsequent image begins to be displayed, during which no image is displayed on the display section or a predetermined interval image is displayed on the display section.

In the image presentation apparatus having the configuration described above, the display controlling section may control the timing at which the subsequent image is displayed on the display section based on a signal value acquired by the biosignal acquisition section during the interval period.

In the image presentation apparatus having the configuration described above, the display controlling section may control the timing at which the subsequent image is displayed on the display section based on a signal value acquired during a display period in which the image is displayed on the display section and the signal value acquired by the biosignal acquisition section during the interval period.

In the image presentation apparatus having the configuration described above, the display controlling section may control the timing at which the subsequent image is displayed on the display section by weighting control based on the signal value acquired by the biosignal acquisition section during the interval period more heavily than control based on the signal value acquired during the display period in which the image is displayed on the display section.

The image presentation apparatus having the configuration described above may further include a biosignal information storage section that stores biosignal information indicating a signal value of a biosignal that indicates a biological response of an observer watching the image in association with the image being watched at a time when the signal value is acquired and an image selecting section that selects a subsequent image to be displayed on the display section based on the signal value acquired by the biosignal acquisition section and the biosignal information stored by the biosignal information storage section. In such a case, the display controlling section may display on the display section the image selected by the image selecting section.

The image presentation apparatus having the configuration described above may further include a biological change information storage section that stores biological change information indicating a change in signal value of a biosignal that indicates a biological response of an observer watching the image in association with the image being watched at a time when the signal value is acquired and an image selecting section that selects a subsequent image to be displayed on the display section based on the signal value acquired by the biosignal acquisition section and the biological change information stored by the biological change information storage section. In such a case, the display controlling section may display on the display section the image selected by the image selecting section.

According to a second aspect related to the innovations herein, one exemplary method may include an image presentation method for presenting an image. The image presentation method includes sequentially displaying a plurality of images, acquiring a signal value of a biosignal indicating a biological response of an observer watching the displayed image, and controlling a timing at which an image subsequent to the image being displayed is displayed based on the acquired signal value.

According to a third aspect related to the innovations herein, one exemplary program may include a program used by an image presentation apparatus that presents an image. The program causes the image presentation apparatus to function as a display section that sequentially displays a plurality of images, a biosignal acquisition section that acquires a signal value of a biosignal indicating a biological response of an observer watching the image displayed on the display section, and a display controlling section that controls a timing at which an image subsequent to the image being displayed on the display section is displayed on the display section based on the signal value acquired by the biosignal acquisition section.

The summary clause does not necessarily describe all necessary features of the embodiments of the present invention. The present invention may also be a sub-combination of the features described above. The above and other features and advantages of the present invention will become more apparent from the following description of the embodiments taken in conjunction with the accompanying drawings.

As made clear from the above description, through the present invention, images can be presented to the observer and switched at an appropriate timing.

### BRIEF DESCRIPTION OF THE DRAWINGS

FIG. 1 is a schematic of an image presentation apparatus 10.

FIG. 2 is a block diagram showing a functional structure of the image presentation apparatus 10.

FIG. 3 shows an operation of the image presentation apparatus 10.

FIG. 4 is a block diagram showing a hardware configuration of the image presentation apparatus 10.

### DESCRIPTION OF EXEMPLARY EMBODIMENTS

Hereinafter, an embodiment of the present invention will be described. The embodiment does not limit the invention according to the claims, and all the combinations of the features described in the embodiment are not necessarily essential to means provided by aspects of the invention.

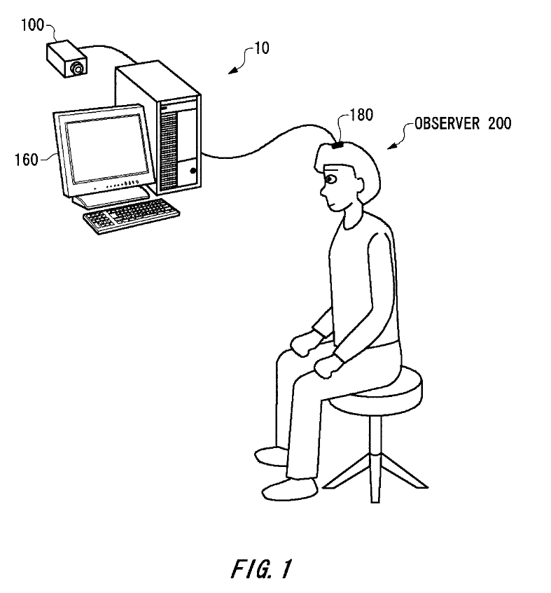

FIG. 1 shows a schematic of an image presentation apparatus 10 according to an embodiment of the present invention. The image presentation apparatus 10 is provided with an image capturing section 100 that captures an image of an observer 200, a biosignal acquisition section 115 that acquires a signal value of a biosignal showing a biological response of the observer 200, a display section 160 that displays an image, and a display controlling section 150 that controls the display section 160 to display the image. The image presentation apparatus 10 displays the image presented to the observer 200 on the display section 160. The image presentation apparatus 10 sequentially changes the image displayed on the display section 160 according to the signal value of the biosignal of the observer 200. In the present invention, the images displayed on the display section 160 include interval images and observational images, described hereinafter.

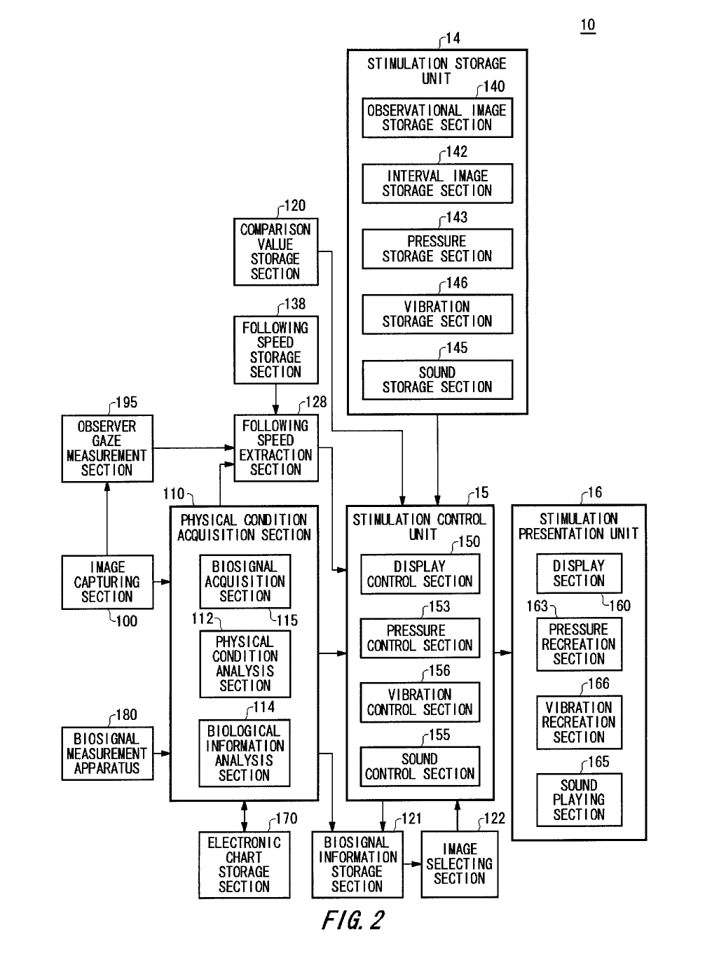

FIG. 2 shows an example of a functional configuration of the image presentation apparatus 10 according to the present embodiment. The image presentation apparatus 10 is provided with an image capturing section 100, a comparison value storage section 120, a biosignal information storage section 121, an image selecting section 122, a physical condition acquisition section 110, a stimulation control unit 15, a stimulation presentation unit 16, a stimulation storage unit 14, a following speed extraction section 128, a following speed storage section 138, an electronic chart storage section 170, a biosignal measurement apparatus 180, and an observer gaze measurement section 195. Furthermore, the physical condition acquisition section 110 includes a biosignal acquisition section 115, a physical condition analysis section 112, and a biological information analysis section 114. The biosignal measurement apparatus 180 may be provided in a separately changeable manner to correspond to the various biosignals without being housed in the image presentation apparatus 10.

The stimulation storage unit 14 includes an observational image storage section 140, an interval image storage section 142, a pressure storage section 143, a vibration storage section 146, and a sound storage section 145. Furthermore, the stimulation control unit 15 includes a sound control section 155, a display controlling section 150, a vibration control section 156, and a pressure control section 153. Furthermore, the stimulation presentation unit 16 includes a vibration recreation section 166, a pressure recreation section 163, a display section 160, and a sound playing section 165.

The observational image storage section 140 stores a plurality of observational images in association with image identification information allocated for every observational image to identify the plurality of observational images sequentially displayed on the display section 160. Here, the observational images are images displayed to affect the mental state of the observer. For example, the observational images may be images that are deeply personal to the observer 200. Such personal images may be, for example, an image of the observer 200 himself or an image taken by the observer 200 himself. In a case where the personal images are presented to the observer 200, the mental state of the observer 200 can be controlled relatively easily because personal images tend to have a great impact on the mental state of the observer 200.

Furthermore, the observational image storage section 140 may store as the observational images 3-dimensional images thought to impart a stronger stimulation to the observer 200 or 2-dimensional images thought to impart a smaller stimulation to the observer 200 than the 3-dimensional images. In a case where 3-dimensional images are stored as the observational images, the observational image storage section 140 may store a plurality of observational images that jump out at the observer to different degrees.

The sound storage section 145 stores the sounds corresponding to the observational images in association with the image identification information. Specifically, in a case where an image of a crowded train is stored in the observational image storage section 140 as the observational image, the sound storage section 145 stores sounds occurring inside a crowded train, such as the sound of the train running, voices of passengers, and announcements by a conductor. The observational image storage section 140, under control of the display controlling section 150, supplies to the display controlling section 150 the observational images to be displayed in the display section 160. The sound storage section 145, under control of the sound control section 155, supplies to the sound control section 155 the sounds associated with the observational images to be displayed on the display section 160. Furthermore, the sound storage section 145 may store sounds that create a feeling that the source of the sound is moving by using a Doppler effect, surround sound, or a change in volume.

The vibration storage section 146 stores the vibrations corresponding to the observational images in association with the image identification information. Specifically, in a case where an image of a crowded train is stored in the observational image storage section 140 as the observational image, the vibration storage section 146 stores the vibration felt on a train associated with the image identification information of the observational image of the crowded train. The vibration storage section 146, under control of the vibration control section 156, supplies to the vibration control section 156 the vibration linked to the observational image to be displayed on the display section 160.

The pressure storage section 143 stores the pressures corresponding to the observational images in association with the image identification information. Specifically, in a case where an image of a crowded train is stored in the observational image storage section 140 as the observational image, the pressure storage section 143 stores the pressure felt by the press of surrounding passengers while riding a crowded train associated with the image identification information of the observational image of the crowded train. The pressure storage section 143, under control of the pressure control section 153, supplies to the pressure control section 153 the pressure associated with the observational image to be displayed on the display section 160.

The interval image storage section 142 stores interval images that are displayed on the display section 160 from a time when an observational image stops being displayed on the display section 160 to a time when the next observational image is displayed. It is desirable that the interval images be images having contents that do not affect the biological response. For example, the interval image storage section 142 may store images having a specified pattern such as a grey screen, an X mark, or the like as interval images.

The image capturing section 100 captures an image of the observer 200 watching the observational images displayed on the display section 160 and an image of the observer 200 during the interval period after the observer 200 has watched the observational images. The image capturing section 100 captures an image of the observer 200 with a resolution sufficient to identify an expression of the observer 200, identify a change in pupil size of the observer 200, and capture an image of the movement of the observer 200. Furthermore, the image presentation apparatus 10 may be provided with a plurality of image capturing sections 100. In addition, the image capturing section 100 may be a 3-dimensional camera or an omnidirectional camera. The image capturing section 100 provides the captured image of the observer 200 to the physical condition analysis section 112 included in the physical condition acquisition section 110.

The physical condition acquisition section 110 acquires a physical condition of the observer 200 watching the observational images displayed on the display section 160 and a physical condition of the observer 200 during the interval period after the observer 200 has watched the observational images. Specifically, the biosignal acquisition section 115 included in the physical condition acquisition section 110 acquires the signal value of the biosignal indicating the biological response of the observer 200 from the biosignal measurement apparatus 180 as the physical condition of the observer 200. The biosignal acquisition section 115 according to the present embodiment acquires a signal indicating an amount of cerebral blood flow. Specifically, the biosignal measurement apparatus 180 of the present embodiment is a cerebral blood flow measurement apparatus that uses a near-infrared blood flow measurement, and the biosignal acquisition section 115 acquires from the biosignal measurement apparatus 180 a signal value indicating a value amount of oxygenated hemoglobin in the brain as the biosignal indicating the amount of cerebral blood flow. The amount of oxygenated hemoglobin is closely related to the level of brain activity, and therefore may be used as an indicator of a case where the brain activity of the observer 200 is controlled to be a prescribed state.

The biosignal acquisition section 115 may acquire other types of biosignals from the biosignal measurement apparatus 180 that measures biosignals indicating, for example, heart rate, blood pressure, breathing depth, body temperature, brain waves, magnetic fields generated by the brain, perspiration, or salivary components of the observer 200. Provided as examples of the biosignal measurement apparatus 180 measuring the aforementioned biosignals are a pulse monitor, a blood pressure monitor, a breathing depth measurement apparatus, a body temperature monitor, an electroencephalograph, a magnetoencephalograph, a magnetic resonance imaging (MRI) scanner, a perspiration monitor, or a salivary component analysis apparatus.

The biosignal acquired by the biosignal acquisition section 115 is not limited to a single type, and the biosignal acquisition section 115 may acquire a plurality of biosignal types. The biosignal acquisition section 115 supplies the signal value of the acquired biosignal to the display controlling section 150.

The physical condition acquisition section 110 may acquire an amount of change in the biosignal acquired by the biosignal acquisition section 115 as the physical condition of the observer 200. Specifically, the biological information analysis section 114 included in the physical condition acquisition section 110 acquires the signal value of the biosignal of the observer 200 from the biosignal acquisition section 115. For example, the image presentation apparatus 10 may show an image that does not stimulate the observer 200 on the display section 160. In such a case, the biological information analysis section 114 acquires the signal value of the biosignal of the observer 200 from the biosignal acquisition section 115 as the signal value of a biosignal at a time when the observer 200 is calm.

Next, the image presentation apparatus 10 displays an observational image on the display section 160. The biological information analysis section 114 compares the signal value of the biosignal of the observer 200 watching the observational image to the signal value of the biosignal of the calm observer 200 and acquires the difference between the two signal values as the physical condition of the observer 200.

Furthermore, the physical condition acquisition section 110 may acquire the physical condition of the observer 200 by causing the physical condition analysis section 112 stored therein to analyze the captured image of the observer 200 received from the image capturing section 100. For example, the physical condition analysis section 112 may detect movement of the observer 200 from a plurality of captured images captured by the image capturing section 100 at predetermined intervals. Next, the physical condition analysis section 112 makes a judgment as to whether the detected movement of the observer 200 corresponds to one of any predetermined movements.

The predetermined movements may include a change of expression (such as from a smile to an indifferent expression), presence or lack of blinking, frequency of blinking, pupil size, averting of the eyes, closing of the eyes, recoiling of the body, turning of the head, hanging of the head, trembling, or the like of the observer 200. For example, the physical condition analysis section 112 may extract the amount of facial area shown by the observer 200 and make a judgment as to whether the observer 200 is hanging their head based on a change in a surface area of the extracted facial area. The physical condition analysis section 112 may calculate the physical condition based on the detected movement.

In addition, the physical condition analysis section 112 may acquire the amount of change per unit of time of the movement of the observer 200 by analyzing the captured images of the observer 200 received from the image capturing section 100. The amount of change per unit of time of the movement of the observer 200 may be, for example, an amount of change per unit of time in the number of times the observer 200 blinks, an amount of change per unit of time in the pupil size, or an amount of change per unit of time in the position of the gaze of the observer 200. Specifically, the physical condition analysis section 112 receives the captured image of the observer 200 from the image capturing section 100 at prescribed times. The physical condition analysis section 112 then analyzes each image of the plurality of received captured images and calculates the amount of change per unit of time of the movement of the observer 200 as the physical condition.

For example, the image presentation apparatus 10 may first display on the display section 160 an image that does not stimulate the observer 200. In such a case, the physical condition analysis section 112 analyzes the captured images received from the image capturing section 100 and calculates the number of times the observer 200 blinks per unit of time while the observer 200 is calm. Next, the image presentation apparatus 10 presents an observational image to the observer 200. In such a case, the physical condition analysis section 112 analyzes the captured images received from the image capturing section 100 at prescribed times and calculates the number of times the observer 200 blinks per unit of time. The physical condition analysis section 112 then compares the number of times the observer 200 blinks per unit of time while the observer 200 is calm to the number of times the observer 200 blinks per unit of time while the observer 200 watches the observational image and calculates the amount of change in the number of times the observer 200 blinks per unit of time. The physical condition analysis section 112 supplies the acquired movement of the observer 200 and the amount of change in the movement of the observer 200 per unit of time to the display controlling section 150 as the physical condition of the observer 200.

In addition, the physical condition acquisition section 110 supplies to the electronic chart storage section 170 the movement of the observer 200 acquired by the physical condition analysis section 112 and the amount of change in the movement of the observer 200 per unit of time. The physical condition acquisition section 110 further supplies to the electronic chart storage section 170 the signal value of the biosignal of the observer 200 acquired by the biological information analysis section 114 and the amount of change in the signal value of the biosignal of the observer 200 per unit of time. The electronic chart storage section 170 stores the signal values of the biosignals and movements of the observer 200 in association with the time. The electronic chart storage section 170, under control of the physical condition acquisition section 110, may supply the signal value of the biosignal and the movement of the observer 200 to the physical condition analysis section 112 and the biological information analysis section 114. For example, the physical condition analysis section 112 may detect the change in movement of the observer 200 by comparing past movement of the observer 200 received from the electronic chart storage section 170 to movement of the observer 200 acquired by analyzing the captured images received from the image capturing section 100. Furthermore, the biological information analysis section 114 may detect the amount of change in the signal value of the biosignal by comparing a past signal value of the biosignal of the observer 200 received from the electronic chart storage section 170 to signal values of the biosignals of the observer 200 received from the biosignal acquisition section 115.

The comparison value storage section 120 stores a comparison value to which the signal value of the biosignal acquired by the biosignal acquisition section 115 is compared at a time when a timing at which the next image is to be displayed on the display section 160 is determined. The comparison value stored by the comparison value storage section 120 is a value corresponding to an expected mental condition of the observer 200. In other words, the comparison value is the expected value at a time when the mental state of the observer 200 is controlled. The comparison value may be set in advance by a user. The comparison value storage section 120 according to the present embodiment stores as the comparison values a first comparison value and a second comparison value, which is greater than the first comparison value.

The comparison value stored by the comparison value storage section 120 may be a single value at a single point in time. Furthermore, the comparison value stored by the comparison value storage section 120 may be a value that is constant over time. Yet further, the comparison value stored by the comparison value storage section 120 may be a value that changes over time. For example, the comparison value storage section 120 may store a comparison value that recreates the change in the biosignal of a case of deep sleep. In such a case, the observer 200 can be effectively led into a deep sleep because the display controlling section 150 can control the timing at which the observational images are presented to cause the signal value of the biosignal to reach the aforementioned comparison value. The comparison value storage section 120 may store a plurality of change patterns of the comparison value or the change pattern may be selected by the user according to a goal.

The display controlling section 150 controls the timing at which the observational image displayed on the display section 160 is changed to the next observational image according to the signal value of the biosignal of the observer 200 received from the biosignal acquisition section 115. For example, where the signal value acquired by the biosignal acquisition section 115 is compared to the comparison value stored in the comparison value storage section 120, the display controlling section 150 may supply to the display section 160 a timing signal indicating a time at which to display the next observational image on the display section 160 based on the a result of the comparison. Specifically, the display controlling section 150 may supply to the display section 160 a timing signal indicating a time at which to display the next observational image on the display section 160 at a time when the signal value acquired by the biosignal acquisition section 115 reaches the comparison value. Furthermore, the display controlling section 150 may supply to the display section 160 a timing signal indicating a time at which to display the next observational image on the display section 160 at a time when the signal value acquired by the biosignal acquisition section 115 is only separated from the comparison value by a prescribed value.

In a case where the signal value acquired by the biosignal acquisition section 115 decreases to reach the first comparison value stored in the comparison value storage section 120 or a case where the signal value acquired by the biosignal acquisition section 115 increases to reach the second comparison value stored in the comparison value storage section 120, the display controlling section 150 of the present embodiment displays the next observational image on the display section 160. Therefore, in a case where the signal value of the biosignal attempts to go beyond the range between the first comparison value and the second comparison value, the display controlling section 150 displays the next image on the display section 160 to keep the signal value of the biosignal within the range between the first comparison value and the second comparison value.

Furthermore, in a case where the signal value acquired by the biosignal acquisition section 115 remains within the range between the first comparison value and the second comparison value for a predetermined prescribed amount of time, the display controlling section 150 may display the next observational image on the display section 160. Therefore, continuous display of an observational image having only a minimal effect in terms of causing the signal value of the biosignal to near the range between the first comparison value and the second comparison value can be avoided.

The display controlling section 150 of the present embodiment has an interval period, which spans from a time when the display of the current observational image is finished to a time when the next observational image is displayed, during which observational images stored in the observational image storage section 140 are not presented to the observer 200. In other embodiments, the display controlling section 150 may display the next observational image immediately after the display of the current observational image is finished without having an interval period.

For example, during the interval period, the display controlling section 150 of the present embodiment displays on the display section 160 the interval images stored in the interval image storage section 142. As an example of another method, during the interval period, the display controlling section 150 may stop the display of any images on the display section 160. Furthermore, during the interval period, the display controlling section 150 may block the display section so that the observer 200 cannot see the display section 160.

The display controlling section 150 of the present embodiment finishes display of the observational image when a prescribed amount of time has passed since the observational image began to be displayed on the display section 160. After the display of the observational image is finished, the display controlling section 150 displays on the display section 160 the interval image stored by the interval image storage section 142. The prescribed time from a time when the observational image begins to be displayed on the display section 160 until a time when the interval image is displayed can be arbitrarily set. In the present embodiment, the prescribed time is set to six seconds. As an example of another method, the display controlling section 150 may finish display of the observational image and then display on the display section 160 the interval image stored by the interval image storage section 142 according to the biosignal acquired by the biosignal acquisition section 115 during display of the observational image.

The response speed, response timing, and the like of the biosignal are different depending on the type of biosignal. For example, the amount of oxygenated hemoglobin in the brain tends to undergo a change after the observer 200 has stopped watching the observational image. Furthermore, brain waves tend to react in as little as six seconds from when the observer 200 begins watching the observational image. According to such different response characteristics depending on the type of biosignal, the biosignal acquisition section 115 acquires at least one of the biosignal during the display period in which the observational image is displayed on the display section 160 or the biosignal during the interval period in which the observational image is not displayed on the display section 160.

The biosignal acquisition section 115 of the present embodiment acquires as the biosignal the amount of oxygenated hemoglobin in the brain, so that the signal value of the biosignal tends to change after the observer 200 stops watching the observational image. Therefore, the display controlling section 150 of the present invention displays the next observational image on the display section 160 based on the signal value of the biosignal acquired by the biosignal acquisition section 115 during the interval period. In a case where the timing for presenting the next observational image is controlled based on a different type of biosignal, the display controlling section 150 may display the next observational image on the display section 160 based on the signal value of the biosignal acquired by the biosignal acquisition section 115 during the display period.

Furthermore, the display controlling section 150 may set the length of the interval period to be a constant time regardless of the signal value of the biosignal acquired by the biosignal acquisition section 115. In such a case, the display controlling section 150 controls the timing at which the change from the display period to the interval period occurs.

Furthermore, the display controlling section 150 may display the next observational image on the display section 160 based on both the signal value of the biosignal acquired by the biosignal acquisition section 115 during the display period and the signal value of the biosignal acquired by the biosignal acquisition section 115 during the interval period. In such a case, the display controlling section 150 may control the time at which the next observational image is displayed according to a weighted combination of the signal value of the biosignal acquired by the biosignal acquisition section 115 during display of the observational image on the display section 160 and the signal value of the biosignal acquired by the biosignal acquisition section 115 during the interval period.

For example, in a case where the signal value acquired by the biosignal acquisition section 115 is controlled to be kept within the range between the first comparison value and the second comparison value, which is larger than the first comparison value, stored by the comparison value storage section 120, the display controlling section 150 may control the timing at which the next observational image is displayed, in a case where the signal value of the biosignal acquired during the interval period is to be weighted more heavily, by setting the first comparison value and the second comparison value during the interval period to be values obtained by multiplying the first comparison value stored in the comparison value storage section 120 by a first coefficient greater than one and multiplying the second comparison value stored in the comparison value storage section 120 by a second prescribed coefficient. In other words, the range between the first comparison value and the second comparison value during the interval period becomes substantially narrower than the range between the first comparison value and the second comparison value during the period in which the observational image is shown.

As another example, in a case where

the signal value acquired by the biosignal acquisition section 115 is outside of the range between the first comparison value and the second comparison value for a predetermined period of time and the next image is controlled to be displayed on the display section 160, the display controlling section 150 may set the prescribed period of the interval period to be shorter than the prescribed period of the display period in a case where the signal value of the biosignal acquired during the interval period is to be weighted more heavily. In other words, the signal value acquired by the biosignal acquisition section 115 is permitted to be outside the range between the first comparison value and the second comparison value for a shorter time during the interval period than during the display period.

The biosignal information storage section 121 stores the biosignal information indicating a characteristic amount of the acquired signal value of the biosignal in association with the image identification information displayed during the display period in which the signal value of the biosignal is acquired or the display period directly before the interval period in which the signal value of the biosignal is acquired. For example, the biosignal information storage section 121 may store as the biosignal information at least one of the signal value of the biosignal indicating a biological response of the observer 200 during the display period or the signal value of the biosignal indicating a biological response of the observer 200 during the interval period. Furthermore, the biosignal information storage section 121 may store as the biosignal information an amount of change in the signal value of the biosignal per unit of time, a maximum or minimum signal value, and a period during which the signal value is to be kept within a prescribed signal value range in association with the image identification information.

The biosignal information storage section 121 may measure in advance and store the characteristic amount of the signal value of the biosignal of the observer 200 corresponding to each observational image in the plurality of observational images. Furthermore, the biosignal information storage section 121 may update and store the characteristic amount of the signal value of the biosignal of the observer 200 corresponding to each observational image in the plurality of observational images for every presentation of an observational image on the display section 160.

The image selecting section 122 selects the next observational image to be displayed on the display section 160 based on the signal value acquired by the biosignal acquisition section 115 and the biosignal information stored by the biosignal information storage section 121. For example, in a case where the signal value acquired by the biosignal acquisition section 115 is a value less than the first comparison value, the image selecting section 122 may select an observational image that has a positive value for the amount of change per unit of time. Furthermore, in the aforementioned case, the image selecting section 122 may select an observational image that has a signal value of the biosignal indicating the biological response of the observer 200 that is between the first comparison value and the second comparison value.

In an embodiment in which the image selecting section 122 selects the next observational image to be displayed on the display section 160, the display controlling section 150 may display on the display section 160 the observational image selected by the image selecting section 122. Furthermore, in an embodiment in which the image selecting section 122 does not select the next observational image to be displayed on the display section 160, the display controlling section 150 may display the observational images stored in the observational image storage section 140 on the display section 160 in a random order.

The display section 160 displays an image under control of the display controlling section 150. Here, a display apparatus such as a monitor or a stereoscopic television is an example of the display section 160. The sound playing section 165 plays the sounds corresponding to the observational image displayed on the display section 160 under control of the sound control section 155. The image presentation apparatus 10 may include a plurality of sound playing sections 165. In a case where the image presentation apparatus 10 includes the plurality of sound playing sections 165, the realistic feel of the observational image shown on the display section 160 can be made greater than in the image presentation apparatus 10 including only a single sound playing section 165.

The vibration recreation section 166 recreates and supplies to the observer the vibrations corresponding to the observational image displayed on the display section 160 under control of the vibration control section 156. For example, the vibration recreation section 166 supplies the vibrations to the observer 200 by shaking a support platform 1660 supporting the observer 200. Specifically, where the support platform 1660 is a chair on which the observer 200 is sitting, the vibration recreation section 166 may shake the chair. Furthermore, where the support platform 1660 is a floor of the room used to examine the observer 200, the vibration recreation section 166 may shake the room itself.

The pressure recreation section 163 recreates and supplies to the observer 200 the pressure corresponding to the observational image displayed on the display section 160 under control of the pressure control section 153. The pressure recreation section 163 may be clothing that houses balloons to be worn by the observer 200. Furthermore, the pressure recreation section 163 may be balloons disposed in the room used to examine the observer 200. In both of the above cases, a high feeling of reality can be achieved by inflating the balloons to provide the observer 200 with the pressure corresponding to the observational image displayed on the display section 160.

The observer gaze measurement section 195 detects the direction of the gaze of the observer 200 based on images of the observer 200 captured by the image capturing section 100. The display controlling section 150 may display the observational image in a position of the display section 160 to be within the gaze of the observer 200 detected by the observer gaze measurement section 195. Furthermore, the display controlling section 150 may cause the position of the observational image to follow the gaze of the observer 200.

The following speed storage section 138 stores the speed at which the image displayed on the display section 160 follows the gaze of the observer 200 in association with the physical condition of the observer 200. Furthermore, the following speed storage section 138 may store the speed at which the image displayed on the display section 160 follows the gaze of the observer 200 in association with the movement of the observer 200, which is the physical condition of the observer 200, or with the amount of change in the movement of the observer 200 per unit of time.

Furthermore, the following speed storage section 138 may store zero (indicating that the display stops moving) as the display following speed in association with the physical condition indicating that the observer 200 is feeling excessive stress. Physical conditions indicating that the observer 200 is feeling excessive stress include, for example, keeping the eyes closed longer than a prescribed time, keeping the eyes averted from the observational image longer than a prescribed time, or the change in the heart rate exceeding a prescribed value.

The following speed storage section 138 may then store the speed at which the observational image displayed on the display section 160 follows the gaze of the observer 200 in association with the biological information of the observer 200, which is the physical condition of the observer 200, or with the amount of change in the biological information of the observer 200 per unit of time. The following speed storage section 138 supplies to the display controlling section 150 the speed at which the observational image follows the gaze of the observer 200 under control of the following speed extraction section 128.

The following speed extraction section 128 extracts the speed at which the observational image follows the gaze of the observer 200 stored by the following speed storage section 138 and associated with the physical condition of the observer 200 acquired by the physical condition acquisition section 110. Specifically, the following speed extraction section 128 extracts the speed at which the observational image follows the gaze of the observer 200 stored by the following speed storage section 138 and associated with the physical condition of the observer 200 acquired by the physical condition analysis section 112 included in the physical condition acquisition section 110.

In addition, the following speed extraction section 128 extracts the speed at which the displayed observational image follows the gaze of the observer 200 stored in the following speed storage section 138 and associated with the movement of the observer 200 acquired by the physical condition analysis section 112 or with the amount of change in the movement of the observer 200 per unit of time. The following speed extraction section 128 may then extract the speed at which the displayed observational image follows the gaze of the observer 200 stored in the following speed storage section 138 and associated with the biological information of the observer 200 acquired by the physical condition analysis section 112 or with the amount of change in the biological information of the observer 200 per unit of time.

The display controlling section 150 displays the observational image on the display section 160 such that the observational image follows the gaze of the observer 200 according to the speed at which the displayed observational image follows the gaze of the observer 200 extracted by the following speed extraction section 128. For example, the following speed storage section 138 stores the speed at which the displayed observational image follows the gaze of the observer 200 in association with the signal value of the biosignal of the observer 200. Specifically, the following speed storage section 138 may store a first following speed in association with a case where the signal value of the biosignal of the observer 200 is less than the first comparison value stored in the comparison value storage section 120. Furthermore, the following speed storage section 138 may store a second following speed, which is slower than the first following speed, in association with a case where the signal value of the biosignal of the observer 200 is within the range between the first comparison value and the second comparison value. Yet further, the following speed storage section 138 may store zero (indicating that the display stops moving) as the display following speed in association with a case where the signal value of the biosignal is greater than or equal to the second comparison value.

The following speed extraction section 128 may then extract the speed at which the displayed observational image follows the gaze of the observer 200 according to the signal value of the biosignal acquired by the biosignal acquisition section 115 and supply the aforementioned speed to the display controlling section 150. The display controlling section 150 displays the observational image on the display section 160 such that the observational image follows the gaze of the observer 200 according to the speed extracted by the following speed extraction section 128.

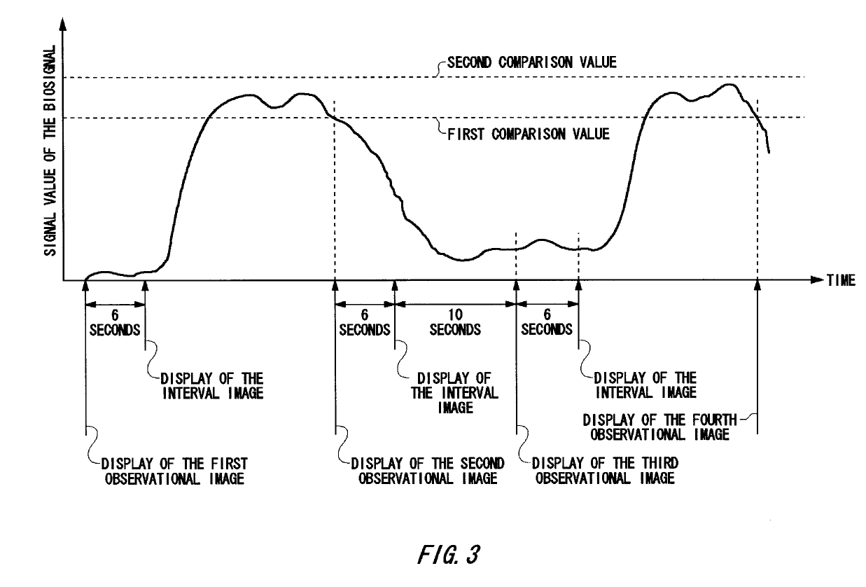

FIG. 3 shows an operation of the image presentation apparatus 10 according to the present embodiment. The display controlling section 150 shows on the display section 160 one of the plurality of observational images (in other words, a first observational image) stored by the observational image storage section 140. The first observational image is selected by the image selecting section 122 from among the plurality of observational images stored in observational image storage section 140 based on the biosignal information stored in the biosignal information storage section 121. Furthermore, the sound control section 155 acquires from the sound storage section 145 the sounds corresponding to the image identification information of the first observational image and causes the sound playing section 165 to play the acquired sounds. The vibration control section 156 acquires from the vibration storage section 146 the vibrations corresponding to the image identification information of the first observational image and causes the vibration recreation section 166 to recreate the acquired vibrations. The pressure control section 153 acquires from the pressure storage section 143 the pressure corresponding to the image identification information of the first observational image and causes the pressure recreation section 163 to recreate the acquired pressure.

When six seconds have passed since the first observational image began to be displayed, the display controlling section 150 stops the display of the observational image on the display section 160 and displays an interval image stored in the interval image storage section 142. Furthermore, the sound control section 155 stops playing the sounds corresponding to the image identification information of the first observational image, the vibration control section 156 stops recreating the vibrations corresponding to the image identification information of the first observational image, and the pressure control section 153 stops recreating the pressure corresponding to the image identification information of the first observational image.

During the interval period after the first observational image has been displayed, the biosignal acquisition section 115 acquires the signal value indicating a value of the amount of oxygenated hemoglobin in the brain from the cerebral blood flow measurement apparatus. The signal value acquired at this time reflects the level of brain activity of the observer 200 watching the first observational image. When the first observational image begins to be shown, the signal value that dropped below the first comparison value is raised to be within the range between the first comparison value and the second comparison value during the interval period. After the signal value is raised, the signal value begins to gradually fall over time until the signal value reaches the first comparison value.

When the signal value reaches the first comparison value, the display controlling section 150 displays the next (a second) observational image on the display section 160. The second observational image is selected by the image selecting section 122 from among the plurality of observational images stored in observational image storage section 140 based on the biosignal information stored in the biosignal information storage section 121.

The biosignal information storage section 121 stores the amount of change in the signal value of the biosignal of the observer 200 watching the first observational image per unit of time, a maximum or minimum signal value, and a period during which the signal value is to be kept within a prescribed signal value range in association with the image identification information of the first observational image. Furthermore, the sound control section 155 acquires from the sound storage section 145 the sounds corresponding to the image identification information of the second observational image and causes the sound playing section 165 to play the acquired sounds. The vibration control section 156 acquires from the vibration storage section 146 the vibrations corresponding to the image identification information of the second observational image and causes the vibration recreation section 166 to recreate the acquired vibrations. The pressure control section 153 acquires from the pressure storage section 143 the pressure corresponding to the image identification information of the second observational image and causes the pressure recreation section 163 to recreate the acquired pressure.

When six seconds have passed since the second observational image began to be displayed, the display controlling section 150 stops the display of the observational image on the display section 160 and displays an interval image stored in the interval image storage section 142. Furthermore, the sound control section 155 stops playing the sounds corresponding to the image identification information of the second observational image, the vibration control section 156 stops recreating the vibrations corresponding to the image identification information of the second observational image, and the pressure control section 153 stops recreating the pressure corresponding to the image identification information of the second observational image.

The biosignal acquisition section 115 acquires the signal value during the interval period after the second observational image has been displayed. The signal value that dropped below the first comparison value after the interval period occurring after the display of the first observational image was completed continues to remain outside the range between the first comparison value and the second comparison value without reaching the first comparison value during the interval period after the second observational image has been displayed. In a case where the signal value has still not entered the range between the first comparison value and the second comparison value after ten seconds have passed since the beginning of the interval period after the display of the second observational image, the display controlling section 150 displays the next (a third) observational image on the display section 160.

The third observational image is selected by the image selecting section 122 from among the plurality of observational images stored in observational image storage section 140 based on the biosignal information stored in the biosignal information storage section 121. The biosignal information storage section 121 stores the amount of change in the signal value of the biosignal of the observer 200 watching the second observational image per unit of time, a maximum or minimum signal value, and a period during which the signal value is to be kept within a prescribed signal value range in association with the image identification information of the second observational image.

Furthermore, the sound control section 155 acquires from the sound storage section 145 the sounds corresponding to the image identification information of the third observational image and causes the sound playing section 165 to play the acquired sounds. The vibration control section 156 acquires from the vibration storage section 146 the vibrations corresponding to the image identification information of the third observational image and causes the vibration recreation section 166 to recreate the acquired vibrations. The pressure control section 153 acquires from the pressure storage section 143 the pressure corresponding to the image identification information of the third observational image and causes the pressure recreation section 163 to recreate the acquired pressure.

When six seconds have passed since the third observational image began to be displayed, the display controlling section 150 stops the display of the observational image on the display section 160 and displays an interval image stored in the interval image storage section 142. Furthermore, the sound control section 155 stops playing the sounds corresponding to the image identification information of the third observational image, the vibration control section 156 stops recreating the vibrations corresponding to the image identification information of the third observational image, and the pressure control section 153 stops recreating the pressure corresponding to the image identification information of the third observational image.

During the interval period after the third observational image has been displayed, the biosignal acquisition section 115 acquires the signal value indicating a value of the amount of oxygenated hemoglobin in the brain from the cerebral blood flow measurement apparatus. The signal value acquired at this time reflects the level of brain activity of the observer 200 watching the third observational image. When the third observational image begins to be shown, the signal value that dropped below the first comparison value is raised to be within the range between the first comparison value and the second comparison value during the interval period. After the signal value is raised, the signal value begins to gradually fall over time until the signal value reaches the first comparison value.

In the same manner as in the interval period after display of the first observational image, the display controlling section 150 displays on the display section 160 the next (a fourth) observational image when the signal value reaches the first comparison value. The fourth observational image is selected by the image selecting section 122 from among the plurality of observational images stored in observational image storage section 140 based on the biosignal information stored in the biosignal information storage section 121.

The biosignal information storage section 121 stores the amount of change in the signal value of the biosignal of the observer 200 watching the third observational image per unit of time, a maximum or minimum signal value, and a period during which the signal value is to be kept within a prescribed signal value range in association with the image identification information of the third observational image. Furthermore, the sound control section 155 acquires from the sound storage section 145 the sounds corresponding to the image identification information of the fourth observational image and causes the sound playing section 165 to play the acquired sounds. The vibration control section 156 acquires from the vibration storage section 146 the vibrations corresponding to the image identification information of the fourth observational image and causes the vibration recreation section 166 to recreate the acquired vibrations. The pressure control section 153 acquires from the pressure storage section 143 the pressure corresponding to the image identification information of the fourth observational image and causes the pressure recreation section 163 to recreate the acquired pressure.

By repeating the process described above, the image presentation apparatus 10 of the present embodiment displays both observational images and interval images while switching therebetween to keep the signal value of the biosignal of the observer 200 in the range between the first comparison value and the second comparison value. As a result, the mental state of the observer 200 can be controlled to be an expected condition.

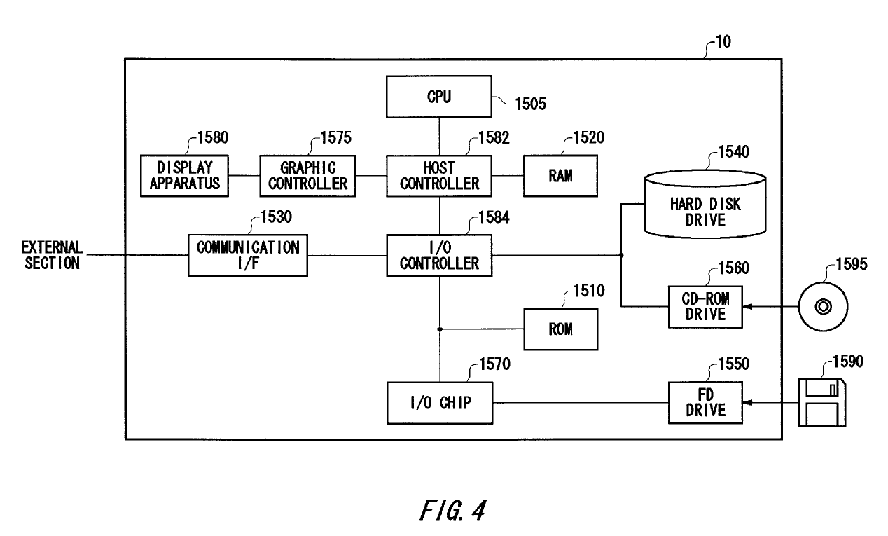

FIG. 4 shows an exemplary hardware configuration of the image presentation apparatus 10 according to the present embodiment. The image presentation apparatus 10 according to the present embodiment is provided with a CPU peripheral section that includes a CPU 1505, a RAM 1520, a graphic controller 1575, and a display apparatus 1580 connected to each other by a host controller 1582; an input/output section that includes a communication interface 1530, a hard disk drive 1540, and a CD-ROM drive 1560, all of which are connected to the host controller 1582 by an input/output controller 1584; and a legacy input/output section that includes a ROM 1510, a flexible disk drive 1550, and a input/output chip 1570, all of which are connected to the input/output controller 1584.

The host controller 1582 is connected to the RAM 1520 and is also connected to the CPU 1505 and graphic controller 1575 accessing the RAM 1520 at a high transfer rate. The CPU 1505 operates to control each section based on programs stored in the ROM 1510 and the RAM 1520. The graphic controller 1575 acquires image data generated by the CPU 1505 or the like on a frame buffer disposed inside the RAM 1520 and displays the image data in the display apparatus 1580. In addition, the graphic controller 1575 may internally include the frame buffer storing the image data generated by the CPU 1505 or the like.

The input/output controller 1584 connects the communication interface 1530 serving as a relatively high speed input/output apparatus, the hard disk drive 1540, and the CD-ROM drive 1560 to the host controller 1582. The communication interface 1530 communicates with other apparatuses via a network. The hard disk drive 1540 stores the programs and data used by the CPU 1505 housed in the image presentation apparatus 10. The CD-ROM drive 1560 reads the programs and data from a CD-ROM 1595 and provides the read information to the hard disk drive 1540 via the RAM 1520.

Furthermore, the input/output controller 1584 is connected to the ROM 1510, and is also connected to the flexible disk drive 1550 and the input/output chip 1570 serving as a relatively high speed input/output apparatus. The ROM 1510 stores a boot program performed when the image presentation apparatus 10 starts up, a program relying on the hardware of the image presentation apparatus 10, and the like. The flexible disk drive 1550 reads programs or data from a flexible disk 1590 and supplies the read information to the hard disk drive 1540 via the RAM 1520. The input/output chip 1570 connects the flexible disk drive 1550 to each of the input/output apparatuses via, for example, a parallel port, a serial port, a keyboard port, a mouse port, or the like.

An image presentation program provided to the hard disk drive 1540 via the RAM 1520 is stored in a storage medium, such as the flexible disk 1590, the CD-ROM 1595, or an IC card, and provided by a user. The image presentation program is read from the storage medium, installed in the hard disk drive 1540 inside the image presentation apparatus 10 via the RAM 1520, and performed by the CPU 1505. The image presentation program installed in the image presentation apparatus 10 are performed by the CPU 1505 to cause the image presentation apparatus 10 to function as the image capturing section 100, the comparison value storage section 120, the biosignal information storage section 121, the image selecting section 122, the physical condition acquisition section 110, the following speed extraction section 128, the following speed storage section 138, the electronic chart storage section 170, the biosignal measurement apparatus 180, the observer gaze measurement section 195, the biosignal acquisition section 115, the physical condition analysis section 112, the biological information analysis section 114, the observational image storage section 140, the interval image storage section 142, the pressure storage section 143, the vibration storage section 146, the sound storage section 145, the sound control section 155, the display controlling section 150, the vibration control section 156, the pressure control section 153, the vibration recreation section 166, the pressure recreation section 163, the display section 160, and the sound playing section 165 described in FIGS. 1 to 3.

While the embodiment of the present invention has been described, the technical scope of the invention is not limited to the above described embodiment. It is apparent to persons skilled in the art that various alterations and improvements can be added to the above-described embodiment. It is also apparent from the scope of the claims that the embodiments added with such alterations or improvements can be included in the technical scope of the invention.

Through the image presentation apparatus, image presentation method and program, images presented to the observer can be switched with an appropriate timing.

What is claimed is:

1. An image presentation apparatus that presents an image, comprising:
a display section that sequentially displays a plurality of images;
a biosignal acquisition section that acquires a signal value of a biosignal indicating a biological response of an observer watching the image displayed on the display section; and
a display controlling section that controls a timing at which an image subsequent to the image being displayed on the display section is displayed on the display section based on the signal value acquired by the biosignal acquisition section.

2. The image presentation apparatus according to claim 1, further comprising:
a comparison value storage section that stores a predetermined comparison value of a biosignal; and
a biosignal value comparison section that compares the signal value acquired by the biosignal acquisition section to the comparison value, wherein the display controlling section controls the timing at which the subsequent image is displayed on the display section based on a comparison result by the biosignal value comparison section.

3. The image presentation apparatus according to claim 2, wherein the display controlling section displays the subsequent image on the display section in a case where the signal value acquired by the biosignal acquisition section reaches the comparison value.

4. The image presentation apparatus according to claim 2, wherein:
the comparison value storage section stores a first comparison value of a biosignal and a second comparison value of a biosignal, which is greater than the first comparison value; and
the display controlling section controls the timing at which the subsequent image is displayed on the display section to keep the signal value acquired by the biosignal acquisition section between the first comparison value and the second comparison value.

5. The image presentation apparatus according to claim 4, wherein the display controlling section displays the subsequent image on the display section in a case where the signal value acquired by the biosignal acquisition section is outside of a range between the first comparison value and the second comparison value.

6. The image presentation apparatus according to claim 5, wherein the display controlling section displays the subsequent image on the display section in a case where the signal value acquired by the biosignal acquisition section is outside of the range between the first comparison value and the second comparison value for a predetermined length of time.

7. The image presentation apparatus according to claim 2, wherein:
the comparison value storage section stores a comparison value for every period of time that passes since the image begins to be displayed on the display section; and
the biosignal value comparison section compares the signal value acquired by the biosignal acquisition section to the comparison value corresponding to the amount of time that has passed since the image began to be displayed on the display section.

8. The image presentation apparatus according to claim 1, wherein the display controlling section has an interval period, which is a period occurring after display of the image is finished and before the subsequent image begins to be displayed, during which no image is displayed on the display section or a predetermined interval image is displayed on the display section.

9. The image presentation apparatus according to claim 8, wherein the display controlling section controls the timing at which the subsequent image is displayed on the display section based on a signal value acquired by the biosignal acquisition section during the interval period.

10. The image presentation apparatus according to claim 8, wherein the display controlling section controls the timing at which the subsequent image is displayed on the display section based on a signal value acquired during a display period in which the image is displayed on the display section and the signal value acquired by the biosignal acquisition section during the interval period.

11. The image presentation apparatus according to claim 10, wherein the display controlling section controls the timing at which the subsequent image is displayed on the display section by weighting control based on the signal value acquired by the biosignal acquisition section during the interval period more heavily than control based on the signal value acquired during the display period in which the image is displayed on the display section.

12. The image presentation apparatus according to claim 1, further comprising:
a biosignal information storage section that stores biosignal information indicating a signal value of a biosignal that indicates a biological response of an observer watching the image in association with the image being watched at a time when the signal value is acquired; and
an image selecting section that selects a subsequent image to be displayed on the display section based on the signal value acquired by the biosignal acquisition section and the biosignal information stored by the biosignal information storage section, and wherein the display controlling section displays on the display section the image selected by the image selecting section.

13. The image presentation apparatus according to claim 1, further comprising:
a biological change information storage section that stores biological change information indicating a change in signal value of a biosignal that indicates a biological response of an observer watching the image in association with the image being watched at a time when the signal value is acquired; and
an image selecting section that selects a subsequent image to be displayed on the display section based on the signal value acquired by the biosignal acquisition section and the biological change information stored by the biological change information storage section, and wherein the display controlling section displays on the display section the image selected by the image selecting section.

14. An image presentation method for presenting an image, comprising;
sequentially displaying a plurality of images;
acquiring a signal value of a biosignal indicating a biological response of an observer watching the displayed image; and
controlling a timing at which an image subsequent to the image being displayed is displayed based on the acquired signal value.

15. A program, used by an image presentation apparatus that presents an image, that causes the image presentation apparatus to function as:
a display section that sequentially displays a plurality of images;
a biosignal acquisition section that acquires a signal value of a biosignal indicating a biological response of an observer watching the image displayed on the display section; and
a display controlling section that controls a timing at which an image subsequent to the image being displayed on the display section is displayed on the display section based on the signal value acquired by the biosignal acquisition section.
